(19) United States
(12) Patent Application Publication
(10) Pub. No.: US 2021/0393450 A1
(43) Pub. Date: Dec. 23, 2021

(54) ARTICLE SIZE CHANGE FORECASTING

(71) Applicant: The Procter & Gamble Company, Cincinnati, OH (US)

(72) Inventor: Blanca ARIZTI, Brooklyn, NY (US)

(21) Appl. No.: 17/346,313

(22) Filed: Jun. 14, 2021

Related U.S. Application Data
(60) Provisional application No. 63/040,668, filed on Jun. 18, 2020.

(51) Int. Cl. A61F 13/42; A61B 5/00; G06Q 30/06; G06N 20/00

(52) U.S. Cl. CPC A61F 13/42; A61B 5/4806; A61F 2013/424; G06Q 30/0631; G06N 20/00; A61B 5/6808

(57) ABSTRACT

A system includes a processor that executes computer executable components. The computer executable components include: a feedback component that receives sensory feedback data from a sensor device in association with wear, by a wearer, of an absorbent article to which the sensor device is attached; and a sizing component that determines whether a size of the absorbent article is appropriate for the wearer based on the sensory feedback data.

### TECHNICAL FIELD

This application relates to sensor systems and more particularly to techniques for automatically determining whether to change the size of a wearer's absorbent article based in part on sensory data captured by the sensor device.

### BACKGROUND

Absorbent articles for personal hygiene, such as disposable diapers for infants, training pants for toddlers or adult incontinence undergarments are designed to absorb and contain bodily exudates, in particular, large quantities of urine. These absorbent articles comprise several layers providing different functions, for example a topsheet, a backsheet and in-between an absorbent core, among other layers. The function of the absorbent core is to absorb and retain the exudates for a prolonged amount of time, for example overnight for a diaper, minimize re-wet to keep the wearer dry and avoid soiling of clothes or bed sheets.

It has been proposed to incorporate sensors into absorbent articles to facilitate sensing usage information (e.g., timing of initiation and level of saturation associated with urination and/or defecation) and/or providing notifications to users (e.g., caregivers, article manufacturers, etc.) regarding the usage information. However, it is believed that improvements are still necessary for such sensors.

Indeed, sensors to date are limited in the information that they obtain and feedback they provide. For example, caregivers are often unaware when their baby has outgrown their diaper, which can lead to leaks, irritation and undesired performance, and known systems fail to address these needs. As such, there is a continued need for sensor systems that provide useful information to caregivers and sensor systems that are incorporated and manufactured with minimal complexity and cost.

### SUMMARY

The invention comprises the features of the independent claims herein. A system comprises a processor that executes the computer executable components. The computer executable components comprise: a feedback component and a sizing component. The feedback component receives sensory feedback data from a sensor device in association with wear, by a wearer, of an absorbent article to which the sensor devise is attached. The sizing component determines whether a size of the absorbent article is appropriate for the wearer based on the sensory feedback data.

A method comprises:

receiving, by a system operatively coupled to a processor, sensory feedback data from a sensor device in association with wear, by a wearer, of an absorbent article to which the sensor device is attached; and

determining, by the system, whether a size of the absorbent article is appropriate for the wearer based on the sensory feedback data.

A machine-readable storage medium comprising executable instructions that, when executed by a processor of a device, facilitate performance of operations, comprising:

receiving sensory feedback data from a sensor device in association with wear, by a wearer, of an absorbent article to which the sensor device is attached;

determining whether a size of the absorbent article is appropriate for the wearer based on the sensory feedback data; and

generating a recommendation regarding changing the size of the absorbent article to a different size based on a determination that the size of the absorbent article is inappropriate for the wearer.

### DESCRIPTION OF THE DRAWINGS

FIG. 1 illustrates a block diagram of an example, nonlimiting system that facilitates forecasting article size changes in accordance with one or more embodiments of the disclosed subject matter.

FIGS. 2A and 2B present a schematic illustration of an example housing of a sensor device in accordance with one or more embodiments of the disclosed subject matter.

FIG. 3 illustrates a block diagram of another example, nonlimiting system that facilitates forecasting article size changes in accordance with one or more embodiments of the disclosed subject matter.

FIG. 4 presents a high-level flow diagram of an example process for evaluating the fit of an absorbent article accordance with one or more embodiments of the disclosed subject matter.

FIG. 5 illustrates a block diagram of another example, nonlimiting system that facilitates forecasting article size changes in accordance with one or more embodiments of the disclosed subject matter.

FIG. 6 illustrates a block diagram of an example external user device that facilitates forecasting article size changes in accordance with one or more embodiments of the disclosed subject matter.

FIG. 7 illustrates a block diagram of an example sensor device that facilitates forecasting article size changes in accordance with one or more embodiments of the disclosed subject matter.

FIG. 8 presents a high-level flow diagram of another example process for determining whether to change the size of absorbent articles accordance with one or more embodiments of the disclosed subject matter.

FIG. 9 presents a high-level flow diagram of another example process for determining whether to change the size of absorbent articles accordance with one or more embodiments of the disclosed subject matter.

### DETAILED DESCRIPTION

The following detailed description is merely illustrative and is not intended to limit embodiments and/or application or uses of embodiments.

“Absorbent article,” as used herein refers to a variety of devices which are placed or worn against or in proximity to the body of the wearer to absorb and contain various exudates discharged from the body, such as disposable diapers. Typically, these absorbent articles comprise a topsheet, a backsheet, an absorbent core and optionally an acquisition system (which may be comprised of one or several layers) and other components, with the absorbent core normally placed between the backsheet and the acquisition system or topsheet. The function of the absorbent core is to absorb and retain the exudates. Although various embodiments of the disclosed subject matter are exemplified in association with the absorbent article being a disposable diaper, it should be appreciated that the disclosed techniques can be applied to a variety of other types of absorbent articles, including reusable diapers (e.g., cloth diapers), absorbent inserts which may be disposable or reusable and may be used in combination with reusable outer covers, pants, training pants, pads, adult incontinence products and/or feminine hygiene products (including, for example, sanitary napkins and tampons).

“Sensor device” refers to any electrical device that can be attached to and/or integrated on or within an absorbent article that provides for capturing and/or generating sensory feedback data associated with wear of the absorbent article via one or more sensors formed on or within the sensor device. In various embodiments, the sensor device can be configured to removably attach to disposable diapers and/or other absorbent articles.

“Sensory feedback data” (or simply “sensory feedback”) as used herein refers to any type of data captured by one or more sensors formed on or within the sensor device and/or determined or inferred based on the captured sensor data. In this regard, unless context warrants particular distinctions among the terms, sensory feedback data can include raw sensor measurements (e.g., raw color sensor data, raw motion sensor measurements, etc.) and/or processed feedback information determined based on the raw sensor measurements using one or more algorithms, heuristics, machine learning models, etc. (e.g., a determined saturation/wetness level, a determined activity level, etc.). Sensory feedback data includes usage data and/or activity information regarding the wearer.

“Usage” in relation to information captured, to be captured, inferred/determined from information captured, processed, detected, stored and/or transmitted, or otherwise used in the sensor systems described herein refers information regarding occurrence and/or timing of the exudation events (e.g., urination, defecation), amount of bodily exudates (e.g., by volume, by weight) associated with an exudation event, saturation levels, time to saturation of the absorbent article, loading status, amount of bodily exudates contained within the absorbent article over a period of time, frequency of exudation events, frequency of article changes, duration of exposure time to bodily exudates, type of the bodily exudates (e.g., urine, feces, discharge, etc.), characteristics of the bodily exudates (e.g., runny bowel, mushy/pasty bowels, viscosity of exudates, coloration of the exudates, etc.), biomarkers present in the bodily exudates, and/or other details related to the use of an absorbent article.

“Activity” in relation to information captured, to be captured, inferred/determined from information captured, processed, detected, stored and/or transmitted, or otherwise used in the sensor systems described herein refers to information regarding exertion levels, movement, exertion and/or movement patterns, sleep/wake patterns, positions, motions, defined movements and motions (e.g., laying, laying on back, laying on stomach, sitting, kicking, rolling, walking, crawling, feeding/suckling, grabbing/pulling on diaper, etc.), and/or other details related to the actions of the wearer during wear of an absorbent article.

“Joined” or “attached”, as used herein, encompasses configurations whereby an element is directly secured to another element by affixing the element directly to the other element, and configurations whereby an element is indirectly secured to another element by affixing the element to intermediate member(s) which in turn are affixed to the other element. The terms further include embodiments in which a pocket or other connector is formed in or attached to an area of the absorbent article. Further, these terms include configurations in which the elements are removably, or non-removably attached.

“Processor” refers to a device or machine that executes machine/computer executable instructions or components stored in memory. A processor as used herein includes, but is not limited to, single-core processors; single-processors with software multithread execution capability; multi-core processors; multi-core processors with software multithread execution capability; multi-core processors with hardware multithread technology; parallel platforms; and parallel platforms with distributed shared memory. Additionally, a processor can refer to an integrated circuit, an application specific integrated circuit (ASIC), a digital signal processor (DSP), a field programmable gate array (FPGA), a programmable logic controller (PLC), a complex programmable logic device (CPLD), a discrete gate or transistor logic, discrete hardware components, or any combination thereof designed to perform the functions described herein. Further, processors can exploit nano-scale architectures such as, but not limited to, molecular and quantum-dot based transistors, switches and gates, in order to optimize use of space or enhance performance of user equipment. A processor can also be implemented as a combination of computing processing units.

“Component” as it relates to a sensor device, a system incorporating a sensor device and/or other machinery herein refers to a computer-related entity or an entity related to an operational machine with one or more specific functionalities. The entities may be hardware, software, a combination of hardware and software, or software in execution. One or more components can reside within a process and/or thread of execution and a component can be localized on one computer and/or distributed between two or more computers. Components can communicate via local and/or remote processes. A component can be an apparatus with specific functionality provided by mechanical parts operated by electric or electronic circuitry, which is operated by a software or firmware application executed by a processor. In such a case, the processor can be internal or external to the apparatus and can execute at least a part of the software or firmware application. As yet another example, a component can be an apparatus that provides specific functionality through electronic components without mechanical parts, wherein the electronic components can include a processor or other means to execute software or firmware that confers at least in part the functionality of the electronic components. In an aspect, a component can emulate an electronic component via a virtual machine, e.g., within a cloud computing system.

“Memory” as used herein refers to mechanism(s) used to retain information, such as executable instructions or components. As used herein, terms such as “store,” “storage,” “data storage,” “database,” and substantially any other information storage element relevant to operation and functionality of a component are utilized to refer to “memory components,” entities embodied in a “memory,” or components comprising a memory. It is to be appreciated that memory and/or memory components described herein can be either volatile memory or nonvolatile memory, or can include both volatile and nonvolatile memory. By way of illustration, and not limitation, nonvolatile memory can include read only memory (ROM), programmable ROM (PROM), electrically programmable ROM (EPROM), electrically erasable ROM (EEPROM), flash memory, or nonvolatile random access memory (RAM) (e.g., ferroelectric RAM (FeRAM). Volatile memory can include RAM, which can act as external cache memory, for example. Additionally, the disclosed memory components of systems or computer-implemented methods herein are intended to include, without being limited to including, these and any other suitable types of memory.

One or more embodiments are now described with reference to the drawings, wherein like referenced numerals are used to refer to like elements throughout. In the following description, for purposes of explanation, numerous specific details are set forth in order to provide a more thorough understanding of the one or more embodiments. It is evident, however, in various cases, that the one or more embodiments can be practiced without these specific details.

### Overview

One or more embodiments of the subject innovation are directed to techniques for determining when an article size change is recommended based on sensory feedback received from a sensor device in association with wear of an absorbent article to which the sensor device is attached. In various embodiments, the sensor device can comprise a device that removably attaches to disposable absorbent articles, and includes one or more sensors configured to capture and/or generate sensory feedback data.

Additionally, or alternatively, the absorbent article can come in various styles and/or types, wherein the different styles or types vary with respect to cut, shape, fit, closures, leg openings/fittings, absorbency level, material (e.g., hypoallergenic materials), type of sensory indicators included, position of the sensory indicators, perfumes and deodorants included, or another feature. With these implementations, the system can include a style/type evaluation component that can evaluate the appropriateness of the current style or type of absorbent article worn by the person and determine, based in part on the sensory feedback data, whether the current style or type of the absorbent article is appropriate. The style/type evaluation component can also determine and recommend an appropriate style/type of absorbent article for wear by the person if the current style/type is determined to be inappropriate.

In various embodiments, one or more algorithms/mathematical models can be developed and applied to determine and/or infer whether the current size and/or style/type of an article is appropriate (e.g., too small or too large) based on the sensory feedback data and/or the additional input information discussed above.

In various embodiments, an application associated with the sensor can receive the sensory feedback and/or the additional input described above and apply the one or more algorithms/mathematical models to the input data to generate notifications regarding recommended article size and/or style/type changes. These and other features are discussed in more detail below.

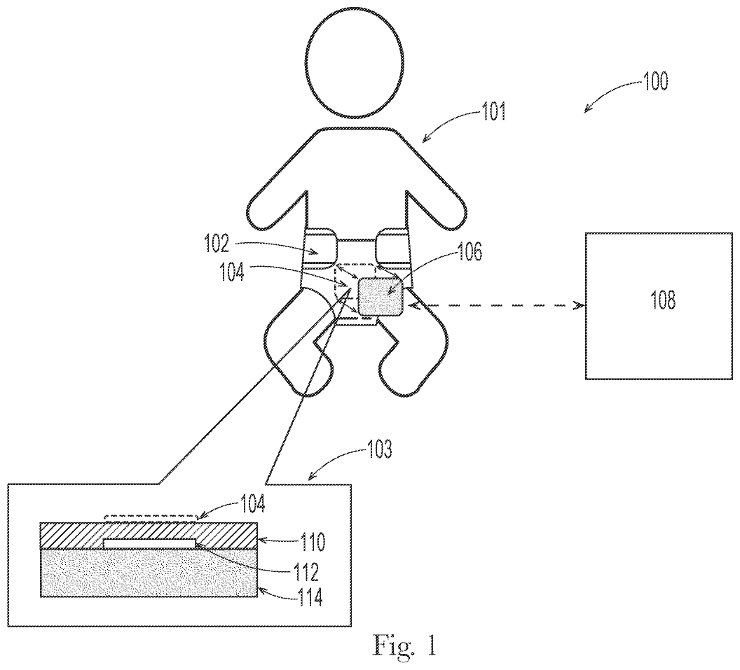

FIG. 1 illustrates a block diagram of an example, nonlimiting system 100 that facilitates forecasting article size changes. Embodiments of systems described herein can include one or more machine-executable components embodied within one or more machines (e.g., embodied in one or more computer-readable storage media associated with one or more machines). Such components, when executed by the one or more machines (e.g., processors, computers, computing devices, virtual machines, etc.) can cause the one or more machines to perform the operations described.

System 100 includes an absorbent article 102 that is worn by a person (i.e., “wearer”) 101, a sensor device 106, and an external user device 108. In the embodiment shown, the absorbent article 102 is a diaper, such as a disposable diaper, that is worn by a growing baby (or toddler, child, etc.). However, it should be appreciated that the absorbent article 102 can be or include a variety of other types of absorbent articles.

In one or more embodiments, the sensor device 106 can be configured to removably attach to the absorbent article 102 and capture and/or generate sensory feedback data associated with wear of the absorbent article 102 by the wearer 101 using one or more sensors formed on or within a housing (described infra) of the sensor device 106. Although system 100 depicts a single absorbent article 102 for use with the sensor device 106, the sensor device 106 can be designed to be reused with and reattached to a plurality of absorbent articles (e.g., disposable diapers). In various embodiments, the sensor device 106 can be configured to removably attach to the absorbent article 102 at or near an attachment zone 104 marked on exterior portion of the absorbent article 102. The location of the attachment zone 104 can vary depending on the type of the absorbent article 102, the type of sensory feedback data the sensor device 106 is configured to capture/detect, and/or the mechanism via which the sensor device 106 captures/detects the sensory feedback data in association with attachment to the absorbent article 102.

For example, in some embodiments, the sensor device 106 can be configured to capture and/or generate sensory feedback data regarding usage and/or activity. In some implementations, the sensory feedback data can include information regarding one or more biomarkers present in the bodily exudates. In various implementations, the sensor device 106 can capture and/or detect information based on responses/reactions reflected in the one or more indicators 112. With these implementations, the absorbent article 102 can comprise one or more indicators 112 formed on or within the absorbent article 102 that generate a response/reaction to indicate certain information (e.g., the presence and/or absence of bodily exudates), and the sensor device 106 can include one or more sensors configured to detect the response/reaction.

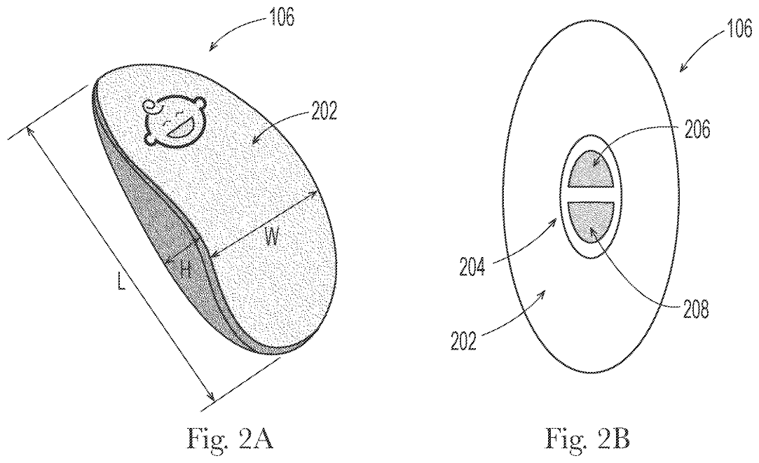

For example, call out box 103 presents an enlarged view of a transversal cross-section of a portion of the absorbent article 102 located directly below the attachment zone 104 in accordance with some example embodiments. As shown in call out box 103, in some embodiments, the absorbent article 102 can comprise a backsheet 110 that can be or correspond to an outer layer of the absorbent article 102. The absorbent article 102 can further include one or more internal layers 114 formed on or joined to the backsheet 110. Such internal layers 114 may include one or more layers of an acquisition system, one or more layers of an absorbent core, and/or one or more layers of the backsheet. The absorbent article 102 can further include one or more indicators 112 formed between the backsheet 110 and the one or more internal layers 114 within a region of the absorbent article that at least partially overlaps the attachment zone 104 to which the sensor device 106 is intended to be attached. For example, in some embodiments, the housing (discussed infra with reference to FIGS. 2A and 2B) of the sensor device 106 can be adapted to be physically coupled to the absorbent article 102 such that the sensor device 106 is further communicatively coupled to the one or more indicators 112 integral with the absorbent article 102.

In some embodiments, at least one indicator 112 may react to usage and/or activity information (or other conditions to be monitored) via one or more changes in a property of the indicator (e.g., a physical, chemical and/or biological property such as color, smell, sound, pH, or the like). By way of nonlimiting example, at least one indicator 112 may react to the presence and/or absence of bodily exudates and/or one or more properties of those bodily exudates as absorbed within the absorbent article. The property or state of the indicator 112, in turn, can be detected by one or more sensors formed on or within a housing of the sensor device 106 when the sensor device 106 is physically and/or communicatively coupled to the absorbent article 102 (e.g., at or near the attachment zone 104). In one particular implementation, the at least one indicator 112 can comprise an optical property changing composition or device (e.g., a color-changing composition or device, such as a color changing indicator) that changes an optical property based on the presence and/or absence of bodily exudates within the one or more internal layers 114 of the absorbent article 102. A color changing indicator can change its color, for example, based on the presence and/or absence of bodily exudates and/or in response to some other condition being monitored with respect to the absorbent article 102.

Essentially any known color-changing indicator that responds to the absence or presence of bodily exudates or other conditions to be monitored with respect to the absorbent article 102 can be used. In some implementations, the absorbent article 102 can employ a color-changing indicator, such as a color strip, which comprises a chemical substance that can induce a color change in the color strip when bodily exudates are present within the one or more internal layers 114. One useful form of a color-changing indicator comprises a pH-sensitive indicator. With these embodiments, the sensor device 106 can include one or more optical sensors, such as a color sensor (also referred to as a colorimeter), to detect information based on detection of a defined color change in the color indicator. For example, the color sensor can provide an output that varies depending on usage information (e.g., the presence or absence of bodily exudates, an amount of the bodily exudates), which is identified/detected by an optical property (e.g., a color) in the color indicator that is observed by the sensor.

In this regard, FIGS. 2A and 2B present a schematic illustration of an example housing 202 for the sensor device 106. FIG. 2A presents a three-dimensional view of top and side surfaces of the housing 202. FIG. 2B presents a two-dimensional view the backside of the sensor device 106, wherein the backside of the sensor device opposes the topside of the housing that includes the baby image/symbol as shown in FIG. 2A, and wherein the backside of the housing 202 is adapted to be attached to and face the attachment zone 104 of the absorbent article.

With reference to FIGS. 2A and 2B in view of FIG. 1, in various embodiments, the housing 202 and/or the absorbent article 102 can include one or more connectors (not shown) for removably attaching the sensor device 106 to the absorbent article 102. The connectors can be provided such that the sensor device 106 can be attached to and detached from the absorbent article 102. In one nonlimiting example, the sensor device 106 can be attached and detached to an area of the absorbent article juxtaposed the indicator integral to the absorbent article 102 (e.g., attachment zone 104).

For example, in various embodiments, the housing 202 and/or the absorbent article 102 can employ various connectors which allow for detachment and can also allow for refastening of the sensor device 106 to the absorbent article 102 at or near the attachment zone 104. In some implementations, the connectors can include one or more adhesives or cohesives formed on the attachment zone 104 and/or on the backside of the housing 202. Such connectors can further include one or more mechanical fasteners, including strap-based fasteners, hook-and-loop-fasteners (e.g., Velcro™), or fasteners comprising at least one button or at least one magnet. In another implementation, a pocket can be formed at or near the attachment zone 104 of the absorbent article 102 and the sensor device 106 can be inserted into the pocket. For example, some absorbent articles can be provided as pants comprising a crotch portion and a belt portion. The crotch portion and the belt portion can be joined adhesively or mechanically. In the area of adhesive joining, a certain portion can be free of adhesive and accessible from the outside. This portion can than serve as a pocket for receiving the sensor device 106. A belt, strap or other device may be used to place and hold the sensor device 106 relative to the absorbent article 102. The sensor device 106 can similarly be joined or held to an article of clothing worn by the wearer of the absorbent article.

In the embodiment shown in FIG. 2B, the sensor device 106 includes a detection unit 204 configured to detect optical property changes reflected in the one or more indicators 112 when the sensor device is attached to the absorbent article at or near the attachment zone. The detection unit 204 may include an optical sensor 206 and a light source 208 (e.g., an LED), which may be located on the backside of the sensor device 106. In accordance with this nonlimiting example, the backsheet 110 of the absorbent article can comprise a transparent or semitransparent material that allows the detection unit 204 to view the optical property changes in the indicator 112 through the backsheet 110. Additionally, or alternatively, one or more indicators 112 can be exposed on or within a region of the backsheet 110 located directly below the attachment zone 104. The backside of the housing 202 can also include a transparent window (e.g., glass, plastic, or another suitable transparent material) or opening through which the optical sensor 206 and the light source 208 are exposed to the environment. In some implementations, the window/opening and/or the optical sensor 206 and light source 208 can further be can be hermetically sealed within the housing 202.

In one or more embodiments, the optical sensor 206 can be configured to measure one or more light levels of a color strip indicator disposed within the absorbent article. In nonlimiting examples, the optical sensor 206 can measure four light levels—clear, red, green and blue—with a sixteen (16) bit resolution. The clear level can correspond to a measure of an overall light intensity and the red, green and blue levels can correspond to intensity in the relevant parts of the spectrum from the color strip indicator. In this embodiment, the sensor device can take multiple measurements with the optical sensor 206. For example, in a first operation, the optical sensor 206 can be read without the light source 208 illuminated to determine a background light level. Another reading of the optical sensor 206 can also be taken in another operation with the light source 208 illuminating the color change indicator to measure the clear, red, green and blue (RGB) light levels. A difference between the two measurements is obtained in a third operation and represents a color of the color strip indicator. The clear color level can be used to normalize the RGB values. Saturation levels corresponding to one or more intermediate states of the color strip indicator can also be determined, such as from the hue, saturation and brightness (HSB) values in combination with or instead of the RGB values.

The optical sensor 206 can be spaced from the light source 208 so that direct light from the light source 208 is reduced or eliminated at the optical sensor 206. Similarly, too large a spacing between the optical sensor 206 and the light source 208 can reduce the signal strength at the optical sensor 206. The optical sensor 206 can be spaced at least about 5.0 millimeters (mm), or at least about 8.0 mm, or at least about 10.0 mm, or from about 5.0 to about 20.0 mm, or from about 10.0 to about 15 mm from the light source 208, reciting for each range every 1 mm increment therein.

In addition to spacing between the optical sensor 206 and the light source 208, other factors may also affect light level measurements of the optical sensor 206. For example, temperature, location of the sensor device 106 on the article, the type, material and color of a connector (e.g., adhesive, tape, hook and loop, strap and other materials) disposed between the sensor device and the indicator, orientation of the sensor device 106 relative to the indicator, orientation of transmit and receive windows of the sensor device 106 and the article, force of application of the sensor device 106 against the article, ambient light, position of an attachment zone 104 on the article and position of the sensor device 106 relative to the indicators 112 within the article (e.g., in a cross-direction) such that the optical sensor 206 detects other portions of the article disposed near the one or more indicators 112.

The size, shape, and/or dimensions of the housing 202 can vary. In some implementations in which the sensor device 106 is designed to be removably attached to disposable diapers, for safety (so as to not become a choking hazard) and convenient handling, the housing 202 can have a length (L) of at least 1.0 centimeters (cm), 2.0 cm, 3.0 cm, 4.0 cm or more (but normally less than 15.0 cm), a width (W) of least 1.0 cm, 2.0 cm, 3.0 cm or more (but normally less than 15.0 cm), and a height (H) of at least 0.5 centimeters, 1.0 cm, 2.0 cm, 3.0 cm, 4.0 cm or more (but normally less than 15.0 cm).

The material employed for the housing 202 can also vary. In some implementations, the housing 202 can be formed with a rigid material (e.g., a rigid plastic). In other implementations, the housing 202 can be formed with a flexible or partially flexible material. To be flexible, the sensor device 106 can incorporate flexible electronic components (and boards). Some suitable materials for the housing 202 can include but are not limited to, silicon, plastic, a thermoplastic, a thermoplastic elastomer (TPE), a confection, a thermosetting polymer, rubber, and the like.

With reference again to FIG. 1, although a pH sensitive color strip indicator is discussed with respect to various example embodiments, indicators 112 are not limited thereto. Rather, indicators 112 can include any indicator that changes color, or another physical property, directly or indirectly related to usage of the article and/or wearer activity. For example, color change materials that change from no color to one or more colors, from one or more colors to no colors, change colors in other color ranges than the pH sensitive adhesive described herein, materials that change color or appearance based on factors other than pH changes, such as but not limited to, temperature, wetness, odor, enzymes, organic components, inorganic components (e.g., salt level), colored SAP/AGM, mechanical forces (e.g., strain, stretch) or the like.

Indicators 112 can also comprise biological or physical sensor materials. For example, physical sensors can be provided by a material, which changes its color when the material is stretched. Stretching of a material can be induced by the swelling of the absorbent core, or other portions, of the absorbent article 102. Biological sensors can include a bioreceptor that interacts with an analyte of interest, such as trypsin or urease. A bioreceptor, for example, can use reagent/analyte interactions that provide a property change (e.g., a color or other optical change) in the absorbent article 102 upon detection of a particular analyte of interest (e.g., a biomarker). Additionally, or alternatively, a bioreceptor can use an immobilized binding reagent capable of binding to an analyte of interest. The immobilized reagent can be disposed on or within one or more layers of the absorbent article 102 adjacent to the attachment zone 104.

Additionally, or alternatively, indicators 112 can comprise a material selected from the group comprising, consisting essentially of or consisting of: thermochromic inks, thermochromic dyes, thermochromic liquid crystalline materials, and combinations thereof. These indicators can, for example, serve to monitor other conditions associated with the absorbent article and/or wearer, such as body temperature or fever indication.

The sensor device 106 can include various other types of sensors that can capture and/or generate sensory feedback data regarding usage of the absorbent article and/or activity of the wearer. The sensor device 106 can include one or more motion sensors (e.g., an accelerometer, a gyroscope, etc.), one or more pressure sensors, one or more temperature sensors, one or more activity sensors (e.g., heart rate monitors, etc.) and the like. For example, the activity information can include motion and/or movement data (e.g., captured via one or more motion sensors) that can be correlated to defined bodily movements and/or positions, movement patterns, activity patterns (e.g., sleep/wake patterns), activity levels, and the like.

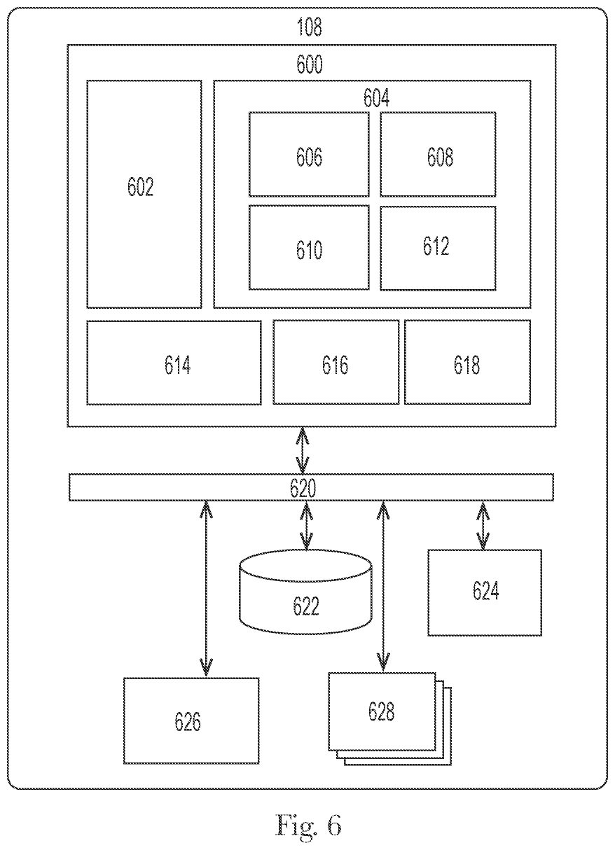

In addition to the one or more sensors, the sensor device 106 can further include suitable electronic circuitry (e.g., hardware), software, or a combination thereof, that provides for processing of raw sensor measurements representative of a measured property (e.g., a wetness/saturation level, an amount of bodily exudates, an activity level, an activity pattern, a defined movement or motion, etc.) as captured via the one or more sensors of the sensor device 106 into a digital signal corresponding to the measured property. For example, such electronic circuitry can include but is not limited to, excitation control elements, amplification elements, analogue filtering elements, data conversion elements, compensation elements, and the like. As described in greater detail infra with reference to FIG. 6, the sensor device 106 can also include or be operatively coupled to at least one memory that stores computer executable instructions and at least one processor (e.g., a microprocessor) that executes the computer executable instructions stored in the memory.

The sensor device 106 can also include suitable communication hardware and/or software that provides for wireless (and/or wired) communication between the sensor device 106 and the least one external user device 108. The external user device 108 can include essentially any type of computing device capable of at least receiving information from the sensor device 106. For example, the external user device 108 can include but is not limited to: a mobile phone, a smartphone, a smartwatch, a tablet personal computer, a laptop computer, a desktop computer, a video monitoring device (e.g., a video baby monitor device), an audio monitoring device (e.g., an audio baby monitor device), an augmented reality (AR) device, a virtual reality (VR), a heads-up display (HUD), a smart speaker device, another sensor device, an IoT device, a television, an Internet enabled television, and similar types of devices.

For example, in some embodiments, the sensor device 106 can be configured to transmit captured sensor data to an external user device 108 associated with a caregiver and/or the wearer 101 (or another suitable entity) for processing and/or presentation to the caregiver etc. via a display, speaker, or another suitable output device of the external user device 108. The external user device 108 can be configured to process and analyze some or all sensor data to determine and/or infer sensory feedback data based on captured sensor measurements. For example, the external user device 108 can receive sensor data from the sensor device 106 identifying or indicating a measured property and/or status of at least one indicator 112. In another example, the external user device 108 can receive sensor data including chemical sensor measurements, temperature sensor measurements, motion sensor measurements, pressure sensor measurements, and the like, as captured via corresponding sensors located on or within the sensor device 106. The external user device 108 can further process/analyze the received sensor data using predefined processing logic (e.g., algorithms, heuristics, machine learning models, defined correlations, tracked data correlations, etc.) to determine and/or infer sensory feedback data regarding usage and/or activity. In some embodiments, the external user device 108 can further present or otherwise render the sensory feedback information at the external user device 108. For example, in implementations, based on a determination that the sensor data indicates the article is wet, the external user device 108 can generate and render a notification at the external user device notifying the caregiver that the article needs changing. In addition to processing and/or rendering the captured sensor data to provide feedback to the user, the sensor device 106 and/or the external user device 108 can also store the sensor data and/or the sensory feedback data determined therefrom in suitable data storage for data aggregation.

In other embodiments, the sensor device 106 itself can include onboard processing logic for processing the sensor data to determine and/or infer sensory feedback data based on the captured sensor data measurements. With these embodiments, the sensor device 106 can be configured to transmit the processed sensory feedback data to the external user device 108 for presentation to the device user and/or for further analytical processing (e.g., by the external user device 108, an application server for the connected care system, another system or the like). For example, the sensor device 106 can include onboard processing logic that can determine when the absorbent article 102 has reached a threshold saturation level and thus requires changing based on a color property or other measured property of color changing wetness indicator provided on or within the absorbent article. Based on a determination that the threshold saturation level has been reached, the sensor device 106 can be configured to transmit a notification message to the external user device 108 that indicates the absorbent article 102 has reached the threshold saturation level. The external user device 108 can further render the notification message at the notification message using an appropriate rendering mechanism (e.g., as a visual notification rendered via a display, as an audible alarm, or the like).

In this regard, the sensor device 106 and the external user device 108 can include suitable communication hardware and/or software that provides for wireless (and/or wired) communication the respective devices. For example, the sensor device 106 and the (at least one) external user device 108 can be communicatively coupled via one or more networks (e.g., a personal area network (PAN), a local area network (LAN), a wide area network (WAN) such as the Internet, and the like). The sensor device 106 and the external user device 108 can employ various suitable wired and/or wireless communication technologies to communicate information therebetween. For example, some suitable communication technologies/protocols can include but are not limited to: Bluetooth®, Bluetooth low energy BTLE®, Mesh (e.g., IEEE 802.15.4), WiFi (e.g., IEEE 802.15.10), communication incorporating all or any portion of IEEE 802 or similar communication standards, RFID technology, near field communication (NFC), 3G communication, 4G communication, 5G communication, Backscatter communication, light communication, audio/sound communication, harvesting protocol communication (e.g., a metadata harvesting protocol), and the like. Other communications protocols or combinations of communications protocols (e.g., a Bluetooth/Mesh combined protocol) can be employed. Additionally, or alternatively, an acoustic or optical broadcasting can be employed. Although system 100 depicts a single external user device 108, it should be appreciated that the sensor device 106 can be configured to communicate with a plurality of external devices of varying types (e.g., user devices, routers, monitors, other sensor devices, server devices, cloud-based systems), etc.

In various embodiments, in addition to or in alternative to using the sensory feedback data captured and/or generated by the sensor device 106 to provide notifications to users regarding usage and/or activity, the sensory feedback data can be used to evaluate the appropriateness of the fit of the absorbent article for the wearer 101. In this regard, caregivers are often unaware when the wearer has outgrown their diaper, which can lead to leaks, irritation and undesired performance. In addition to size, in some implementations, the absorbent article 102 can be made in different styles or types that provide different fits, shapes or cuts, have different absorption levels (e.g., light, medium, heavy, etc.), are formed with different materials (e.g., hypoallergenic materials, sensitive skin materials, etc., and/or provide different features and functionalities (e.g., different types of indicators for detection of different biomarkers present in bodily exudates).

To facilitate determining when a wearer has outgrown their diaper (or a similar type of absorbent article) and/or determining an appropriate size and/or style/type of absorbent article, in various embodiments, the external user device 108 (or another computing device) can be configured to process the sensory feedback data using one or more fit evaluation algorithms/mathematical models. The one or more fit evaluation algorithms/models can include for example, heuristic based algorithms/models, statistical-based algorithms/models, probabilistic-based algorithms models, and/or machine learning algorithms/models configured to generate an output regarding an appropriate size type and/or style of absorbent article 102 for wear by the wearer 101 based at least in part on the sensory feedback data, including for example activity information and/or usage data. For example, in some implementations, the one or more fit evaluation algorithms/models can include one or more algorithms/models configured to evaluate appropriateness of the current size, type or style of the absorbent article for the wearer 101 based at least in part on the sensory feedback data. The one or more fit algorithms/models can also include one or more algorithms/models configured to determine and/or infer an appropriate size, type and/or style of absorbent article 102 (e.g., from amongst a known selection of different sizes, types and/or styles) for wear by the wearer 101 based at least in part on the sensory feedback data. Additionally, or alternatively, the one or more fit algorithms/models can include one or more forecasting models configured to predict when a person will outgrow their current size, style and/or fit of absorbent article in implementations in which their current size, style and/or type of absorbent article is determined to be appropriate. The external user device 108 can further notify a user of the external device (e.g., a caregiver) regarding the results of the one or more fit evaluation algorithms/models.

In this regard, the one or more fit evaluation algorithms/models can be configured to generate an output, based on one or more input parameters included in the sensory feedback data, that indicates whether the size and/or style/type is appropriate. For instance, such input parameters can include usage data parameters regarding usage (e.g., timing of occurrence of the urination/defecation, time to saturation, etc.). For example, in one or more embodiments, the one or more fit evaluation algorithms/models can correlate the amount of bodily exudate received and/or absorbed within the absorbent article to a particular article size (e.g., an amount of bodily exudates between X amount and Y amount indicates an article size of medium is recommended for the wearer). In other nonlimiting examples, the one or more fit evaluation algorithms/models may also factor in the average amount of bodily exudates associated with each urination/defecation event over a defined period of time (e.g., one day, one week, etc.), time to saturation of the article, and/or frequency of article changes to a particular size article considered suitable for the wearer.

The input parameters can also include activity related information (e.g., activity patterns and levels of the wearer, sleep patterns of the wearer). For example, the one or more fit evaluation algorithms/models can be configured to generate an output that indicates whether the current size, type and/or style of article is appropriate based on determined activity patterns and levels of the wearer, specific motions or movements (e.g., kicking, grabbing/bulling on the diaper, etc.), amounts and/or duration of the motions/movements, frequency of performance of the motions/movements, and the like.

In some embodiments, additional input may be entered/reported by the user regarding the wearer and/or fit, hereinafter “user feedback data.” User feedback data can include (but is not limited to): information regarding demographics of the wearer 101 (e.g., current age, gender, etc.), the current size and/or style/type of absorbent article worn, body shape and size (e.g., height, weight, body mass index (BMI), body measurements, etc.), behaviors of the wearer in association with wear and removal of the absorbent articles (e.g., facial expressions, crying, laughing, kicking, grabbing/bulling on the diaper, etc.), mood of the wearer in association with wear and removal of the absorbent article), diet and feeding schedule, information indicating whether the wearer is toilet training, degree of wearer incontinence, article change frequency, observed leakage, observed irritation, and the like. This information about the wearer can also be used as input to the one or more fit evaluation algorithms/models and/or upgrade forecasting algorithms/models to determine if the size is appropriate. These inputs can be reported manually (e.g., via an application associated with the sensor device that is executed by the external user device 108), retrieved from other accessible information systems, and/or captured via one or more external devices/system. For example, in some implementations, additional information about the wearer can include user provided feedback received as a natural language description (e.g., in plain text, as a verbal spoken description, etc.), in association with completion of a survey or questionnaire with predefined selectable responses to questions regarding the wearer, fit, wear marks and the like.

Additional input data can also include image data (e.g., still images and/or video) of the wearer showing how the article fits (e.g., including relative size and positions of parts of the absorbent article to body parts of the person, sagging, position of the adhered side flaps of the diaper, etc.), wear marks on the person's body attributed to the article (e.g., around the waist and legs when the diaper is removed), and the like. In some implementations, image data of the wearer during wear and/or with the absorbent article 102 removed (e.g., to capture wear marks) can be captured using the external user device 108. In other implementations, a video monitoring device can be used in conjunction with the sensor device 106 to capture video and image data of the person for generating feedback for input into the one or more fit evaluation algorithms/models.

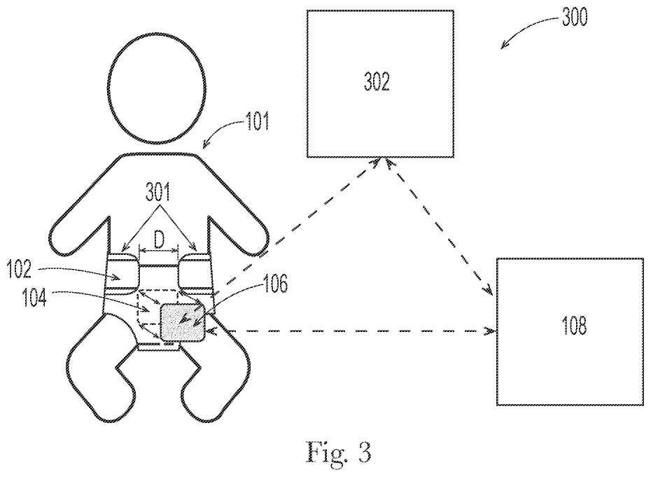

For example, FIG. 3 illustrates a block diagram of another example, nonlimiting system 300 that facilitates forecasting article size changes in accordance with one or more embodiments of the disclosed subject matter. System 300 include same or similar features and functionalities as system 100 with the addition of an auxiliary monitoring device 302. Repetitive description of like elements employed in respective embodiments is omitted for sake of brevity.

In various embodiments, the auxiliary monitoring device 302 can include a video monitoring device that captures video and/or image data of the wearer 101 in association with wear of the absorbent article 102. For example, in implementations in which the wearer 101 is a baby (or toddler, child, etc.), the video data can include video of the baby while located in a crib, while being changed (e.g., diaper change), while playing in a playroom, or the like. In other implementations, the auxiliary monitoring device 302 can be configured to capture still images of the baby in association with wear of the absorbent article 102. The video and/or image data captured by the auxiliary monitoring device 302 can further be used input in association with evaluating the appropriateness of the size, type and/or style of the absorbent article.

Further, in some implementations, the video and/or image data can be processed using image analysis software to evaluate the fit, to identify and characterize wear marks (e.g., around the waist and legs), and the like. For instance, the image analysis software can be configured to evaluate image data of the wearer to determine relative positions of one or more parts of the absorbent article 102 to one another and/or one or more parts of the person's body (e.g., relative locations of the article ears and/or fasteners 301 to the longitudinal center of the article or another midline feature). For example, if the distance D between diaper ears and/or fasteners 301 exceeds a maximum threshold distance, the one or more fit algorithms/models can be configured to classify the diaper as being too large. Likewise, if the distance D between diaper ears/fasteners 301 is less than a minimum threshold distance, the one or more fit algorithms/models can be configured to classify the diaper as being too small. Additionally, or alternatively, the image analysis software can be configured to evaluate image data of the wearer to determine relative proportions of body features to one or more parts of the absorbent article (e.g., a size of the person's thigh relative to a circumference of the leg opening of the diaper, length of the persons torso relative to a length of the diaper, etc.), a degree of sagging of the diaper (e.g., when dry, when wet, after reaching a defined level of saturation, after being saturated for N number of minutes, etc.) and the like. The image data can also be processed using image analysis software to identify and track information such as wearer activity (e.g., movements/motions), wearer behaviors, wearer mood (e.g., based on facial features patterns), and the like, which can also be used as input to the one or more fit evaluation algorithms/models to determine appropriateness of the current article size and/or style/type, to determine a different, more appropriate size and/or style/type, and/or to forecast when a upgrade to a different size and/or style/type is recommended.

The auxiliary monitoring device 302 can further be communicatively coupled to the external user device 108 (and in some implementations, the sensor device 106) via one or more wireless communication technologies described herein. In this regard, in some embodiments, the auxiliary monitoring device 302 can perform some or part of the image analysis to extract the input parameters from the video/image data for input to the one or more fit evaluation algorithms/models. With these embodiments, the auxiliary monitoring device 302 can send the external user device 108 the extracted/identified input parameters. In other embodiments, the auxiliary monitoring device 302 can be configured to send the captured video/image data to the external user device 108 for image analysis processing to extract the relevant input parameters.

In some embodiments, the auxiliary monitoring device 302 can also be or include an audio recording device that captures audio from the wearer 101 in association with wear of the absorbent article 102. With these embodiments, audio analysis software can be employed to determine sounds (e.g., laughing, crying, sleeping, speaking), identify words spoken, determine tone of voice, volume, and the like. The processed sound information can also be used as input to the one or more fit evaluation algorithms/models to evaluate the appropriateness of the size and/or style/fit of the absorbent article 102 for the wearer 101. For example, in some implementations, the frequency, duration and/or tone of detected crying can be correlated to irritation attributed to a diaper fit that is too small for the baby.

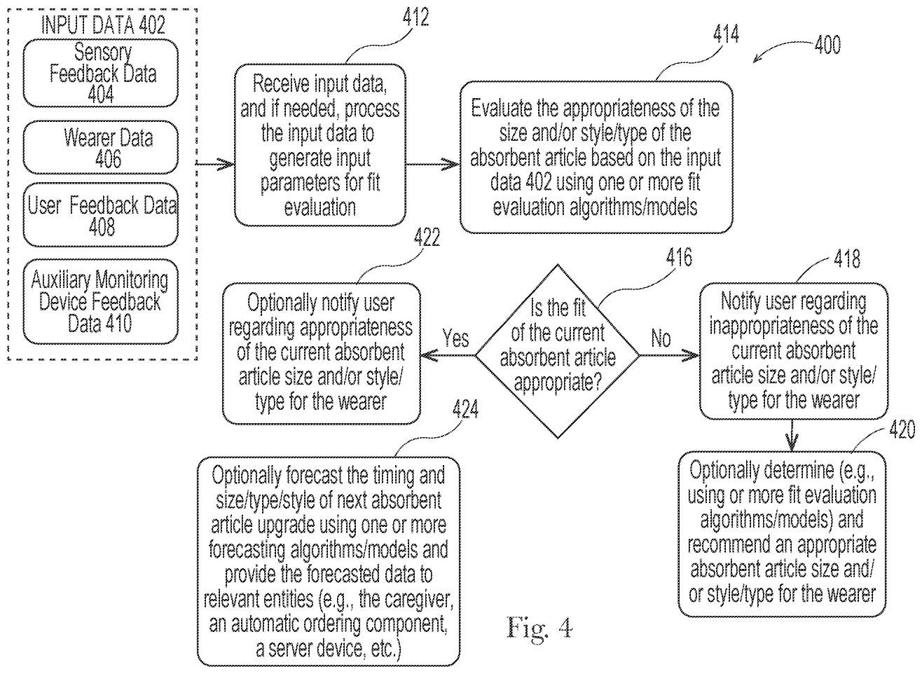

FIG. 4 presents a high-level flow diagram of an example process 400 for evaluating the fit of an absorbent article accordance with one or more embodiments of the disclosed subject matter. With reference to FIG. 4 and FIG. 3, process 400 is described in association with implementation by the external user device 108. However, it should be appreciated that process 400 and/or one or more processing steps of process 400 can be executed by the sensor device 106 and/or another device (e.g., a server device). Repetitive description of like elements employed in respective embodiments is omitted for sake of brevity.

In accordance with process 400, at 412 the external user device 108 can receive input data 402 and if needed, process the input data to generate the input parameters for fit evaluation (e.g., for input into the one or more fit evaluation algorithms/models). In the embodiment shown, the input data 402 can include sensory feedback data 404, wearer data 406, user feedback data 408 and/or auxiliary monitoring device feedback data 410. As described with reference to FIGS. 1 and 3, in various embodiments, the sensory feedback data 404 can include usage data and/or activity data determined based on information captured via one or more sensors of the sensor device 106.

The wearer data 406 can include various relevant statistics about the wearer, such as the current article size worn, age, gender, height, weight, BMI, body measurements and the like. In some implementations, the wearer data 406 can also include information regarding fit preferences of the wearer, diet of the wearer, feeding habits/schedule of the wearer (e.g., liquid diet, semi-solids, timing, etc.), and the like. The user feedback data 408 can include user reported feedback regarding the wearer and/or the fit of the absorbent article. For example, in some implementations, the wearer data 406 described above can be received manually as user input. The user feedback data 408 can also include information regarding current article size, fit, behavior of the wearer in association with wear of the articles, change frequency, whether the wearer is toilet training, degree of wearer incontinence, and the like. The auxiliary monitoring device feedback data 410 can include video data, image data, and/or audio capture of the wearer that can be processed by the external user device 108 and/or has been processed to determine input parameters regarding fit, wear marks, wearer behaviors, wearer gestures, wearer mood and the like.

At 414, the external user device 108 can evaluate the appropriateness of the size and/or style/type of the absorbent article for the wearer based on the input data 402 using one or more fit evaluation algorithms/models. In this regard, the fit evaluation algorithms/model(s) can be configured to generate an output that reflects a measure of appropriateness of the current article size, type and/or style for the wearer based on the input data 402. For example, in some implementations, the fit evaluation algorithms/model(s), such as sizing and/or style/type evaluation algorithms/model(s), can generate a binary output result that classifies the article size and/or style/type as either appropriate or inappropriate, too big or too small, or just right.

In other example embodiments, the one or more fit evaluation algorithms/models can be configured to generate an appropriateness score that reflects a degree or level of appropriateness of the article size and/or style/type for the wearer. For instance, the score can reflect a scale of 1 to 10 wherein a 1 reflects the lowest level of appropriateness and a 10 reflects the highest level of appropriateness. The appropriateness score can be based solely on size, solely on type and/or style, or a combination of size and style/type. Alternatively, at 414, two appropriateness scores can be generated, one that reflect appropriateness of the size of the absorbent article for the wearer, and another that reflects appropriateness of the type of the absorbent article for the wearer. Other suitable scoring scales can be used (e.g., a percentage score out of 100, or another suitable scoring mechanism). In some implementations of these embodiments, a size appropriateness score can be generated that reflects a degree of appropriateness of the size and indicates whether the size is too big or too small. For example, the scoring rubric can employ a sale of −5 to +5, wherein a score of 0 indicates a perfect or most appropriate size, a score of −5 indicates a lowest score of appropriateness in the direction of being too small, and score of +5 indicates a highest score of appropriateness in the direction of being too big.

At 416, the external user device 108 can determine whether the current article size and/or style/type is appropriate for the wearer based on the results of the fit evaluation algorithm/model(s). For example, in implementations in which the fit evaluation algorithm/model(s) only provide an output that reflects appropriateness of the size, at 416, the external user device 108 can determine if the fit of the current absorbent article is appropriate based on whether the size is determined to be appropriate or inappropriate (e.g., too big or too small). Likewise, in implementations in which the fit evaluation algorithms/models only provide an output that reflects appropriateness of the style/type of the absorbent article for the wearer, at 416, the external user device 108 can determine if the fit of the current absorbent article is appropriate based on whether the style/type is determined to be appropriate or inappropriate. In other implementations in which both size and style/type appropriateness is evaluated, at 416, the external user device 108 can determine that fit of the current absorbent article is inappropriate if either the size or the style/type is determined to be inappropriate. In other embodiments, in which an appropriateness score is generated, the external user device 108 can apply a predefined thresholding mechanism to determine if the score renders the article appropriate or inappropriate for the user with respect to size, style/type, or both. For example, if the appropriateness score is above a minimum threshold score, the external user device 108 can classify the current article size and/or style/type as appropriate. Alternatively, if the appropriateness score is below the minimum threshold score, the external user device 108 can classify the current article size and/or style/type as inappropriate.

If at 416, the external user device 108 determines that the fit (i.e., size and/or style/type) of the current absorbent article is inappropriate for the wearer, then at 418 the external user device can notify the user of the external device (e.g., a caregiver) of the inappropriateness of the current size and/or style/type (e.g., via a visual or audible notification rendered at the external user device 108). For example, in some implementations, the external user device 108 can notify the user if the size is determined to be too small or too large and/or provide a size appropriateness score. The external user device 108 can also notify the user that the current style/type is inappropriate and/or provide a style/type appropriateness score. In addition, at 420, the external user device 108 can optionally determine and recommend a more appropriate size and/or style/type article for the wearer using the one or more fit evaluation algorithms/models. For example, the external user device 108 can determine a specific size absorbent article from amongst the available size options that best suits the wearer based on the input data 402. Similarly, the external user device 108 can determine a specific style/type absorbent article from amongst the available style/type options that best suits the wearer based on the input data 402. The external user device 108 may also notify the user of various sizes and/or styles/types available from one or more manufacturers, retailers etc.

If at 416, the external user device 108 determines that the fit of the current absorbent article is appropriate for the wearer, then at 422 the external user device 108 can optionally notify the user of the external device of the appropriateness of the current article size and/or style/type for the wearer. In some implementations in which an appropriateness score is generated at 414, the notification at 422 can also include the appropriateness score. In addition, at 424, the external user device 108 can optionally forecast the timing and size/style/type of next absorbent article upgrade using one or more forecasting algorithms/models and provide the forecasted data to the relevant entities (e.g., the caregiver, an automatic ordering component, the server device, etc.). In nonlimiting examples, the external user device may notify the user of the various sizes and/or styles/types available from one or more manufacturers, retailers etc.

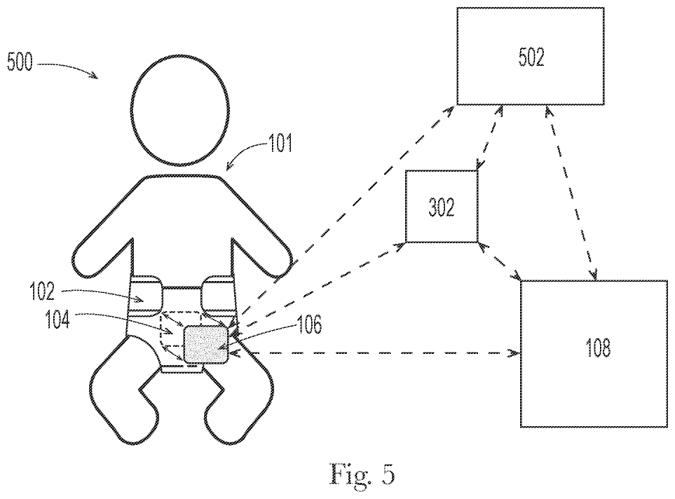

FIG. 5 illustrates a block diagram of another example, nonlimiting system that facilitates forecasting article size changes in accordance with one or more embodiments. System 500 includes same or similar features and functionalities as system 300 with the addition of a server device 502. Repetitive description of like elements employed in respective embodiments is omitted for sake of brevity.

System 500 includes a plurality of distributed devices, including sensor device 106, external user device 108, auxiliary monitoring device 302 and server device 502. One or more of these devices can be communicatively coupled to one another, either directly or indirectly (e.g., via another device) using one or more wired and/or wireless communication technologies described herein. For example, in some embodiments, the server device 502 and the sensor device 106 can be configured to communicate with one another using one or more WAN wireless communication technologies. For instance, the server device 502 can communicate directly with the sensor device 106 to provide firmware updates over the air, to receive sensory feedback data from the sensor device 106, and the like. In other embodiments, the server device 502 and the sensor device can communicate indirectly through the external user device 108 and/or the auxiliary monitoring device 302.

In various embodiments, the server device 502 can be or correspond to one or more cloud-based server devices that can facilitate provision of a variety of ancillary services to customers associated with the sensor device 106 via a mobile device application deployed at the external user device 108 and/or web-based platform (e.g., a website, a web-application, or the like) accessed using the external user device 108. For example, with reference to FIG. 5 in view of FIG. 4, in some implementations, the mobile device application and/or web-based platform system can facilitate processing the input data 402 to evaluate the appropriateness of the size and/or style/type of the absorbent article 101. In this regard, alternative to the this evaluation being performed by the sensor device 106 or the external user device 108, the sensor device 106 and/or the auxiliary monitoring device 302 can send (or otherwise provide) the input data 402 (e.g., the sensory feedback data 404, the wearer data 406, the user feedback data 408 and/or the auxiliary monitoring device feedback data 410) to the server device 502. The server device 502 can further apply fit evaluation algorithms/models to the received input data to determine whether the size and/or style/type of the absorbent article 102 is appropriate for the wearer 101. The server device 502 can also apply fit evaluation algorithms/models to determine an appropriate size and/or style/type of absorbent article for wear by the person. The server device 502 can further apply the input data 402 to one or more forecasting models to forecast when the wearer 101 is likely to outgrow their current size and/or style/type of absorbent article 102 and forecast the next size and/or style/type of absorbent article that will best suit the wearer's needs at that time. The server device can further provide the external user device 108 with information regarding the results of fit evaluation algorithms/models, forecasting models (e.g., in the form of a notification, a report, etc.), and/or various sizes and/or styles/types available from one or more manufacturers, retailers etc.

In some embodiments, the server device 502 can also regularly and/or continuously update fit evaluation algorithms/models based on data collected for a plurality of users over time. In this regard, the server device 502 can receive input data 402 for many different absorbent article wearers over time. For example, the server device 502 can collect input data 402 for many different wearers with similar and dissimilar attributes (e.g., ages, genders, weights, BMIs, diets, feeding schedules, etc.) and that wear similar and dissimilar sizes and/or styles/types of absorbent articles, such as disposable absorbent articles. The server device 502 can further employ one or more machine learning techniques (e.g., supervised, semi-supervised, and/or unsupervised) to update the one or more fit evaluation algorithms/models and/or forecasting models based on the collected input data. In embodiments in which a mobile device application deployed at the external user device 108 applies the one or more fit algorithms/models and/or forecasting models to the input data 402 for a particular person, the server device 502 can further regularly send the external user device 108 updates for the application that include the updates to the one or more fit evaluation algorithms/models and/or forecasting models.

The server device 502 can provide additional ancillary services to customers associated with the sensor device 106 via the mobile device application and/or web-based platform (e.g., a website, a web-application, or the like), including but not limited to, purchasing/ordering additional absorbent articles, purchasing/ordering other authorized products for usage with the sensor device 106, purchasing/ordering additional sensor devices, providing user with rewards (e.g., coupons, discounts, gifts, etc.), providing user with requests/notifications related to authorizing the server device 502 to receive or otherwise access the data collected (e.g., input data 402), purchasing batteries for the sensor device and the like.

FIG. 6 illustrates a block diagram of an example external user device (e.g., external user device 108) that facilitates forecasting article size changes. The external user device 108 can includes several computer executable components that provide various features and functionalities associated with use of the external user device 108 in the context of an absorbent article sensor system, such as system 100, system 300, system 500 and the like. These computer executable components can include feedback component 602, fit evaluation component 604, recommendation component 614, ordering component 616 and reward component 618.

In the embodiment shown, these computer executable components are associated with a connected care application 600. For example, the connected care application 600 can include a dedicated client application, a web-application, a thin client application, a hybrid application, or the like. In this regard, the connected care application 600 can provide various online features and functionalities associated with communication between the connected care application 600 and at least one external server (e.g., server device 502) and/or another suitable networked service providers (e.g., a cloud-based server device, an application server, and the like). In other embodiments, one or more features and functionalities of the connected care application 600 can be performed offline.

It should be appreciated however that the deployment architecture for the feedback component 602, the fit evaluation component 604, the recommendation component 614, the ordering component 616 and/or the reward component 618 can vary. For example, in some embodiments, one or more of these components can be provided with and/or executed by the sensor device 106, the auxiliary monitoring device 302, and/or the server device 502.

In addition to the computer executable components identified above (e.g., the feedback component 602, the fit evaluation component 604, the recommendation component 614, the ordering component 616 the reward component 618), the external user device 108 can include a communication component 626, at least one memory 622, at least one processor 624, one or more cameras 628, and a device bus 620. It should be appreciated that in some embodiments, one or more of the components shown in FIG. 6 can be removed from the external user device 108. In other embodiments, one or more of the components shown in FIG. 6 can executed by the sensor device 106, the server device 502 and/or another computing device. In various embodiments, the at least one memory 622 can be configured to store computer executable components and instructions (e.g., the connected care application 600 and/or one or more components of the connected care application 600). The external user device 108 can also include at least one processor 624 to facilitate operation of the computer executable components and instructions by external user device 108. The external user device 108 can further include a device bus 620 that couples the various components of the external user device 108, including, but not limited to, the connected care application 600, the communication component 626, the memory 622, the processor 624, and the one or more cameras 628.

The communication component 626 can provide for communicatively coupling the external user device 108 with one or more external devices, such as sensor device 106, auxiliary monitoring device 302, server device 502 and/or various other devices remote (e.g., physically remote) from external user device 108. In this regard, the communication component 626 can include software, hardware, or a combination of software and hardware that is configured to facilitate performance of wireless (and/or wired) communications between the external user device 108 and the one or more external devices. For example, the communication component 626 can include and/or be configured to control operation of one or more transmitters/receivers of the external user device 108 to provide for transmitting information to the one or more external devices and/or receiving information from the one or more external devices.

The communication component 626 can be configured to facilitate wireless communication with the one or more external devices (e.g., the sensor device 106, the auxiliary monitoring device 302 and/or the server device 502) using a variety of wireless communication protocols. For example, in one or more embodiments, the communication component 626 can communicate with an external device using a Bluetooth® communication protocol, a near-field communication (NFC) protocol, or another type of communication protocol over a PAN or a LAN, (e.g., a Wi-Fi network) that can provide for communication over greater distances than NFC protocol or that can provide various advantages (such as increased security). Other communication protocols that can be employed by communication component 904 to communicate with an external device can include, but are not limited to: a Session Initiation Protocol (SIP) based protocol, a Zigbee® protocol, a RF4CE protocol, a WirelessHART protocol, a 6LoWPAN (IPv6 over Low power Wireless Personal Area Networks) protocol, a Z-Wave protocol, an ANT protocol, an ultra-wideband (UWB) standard protocol, a cellular communications protocol (e.g., second, third, fourth and fifth Generation Partnership Project (GGP) protocols, Long Term Evolution (LTE), protocols), machine type communication (MTC) protocols, Narrowband Internet-of-things (NB-IoT) protocols, other radio frequency (RF) communication protocols, non-RF communication protocols (e.g., induction based, optical based, audio based, etc.) and/or other proprietary and non-proprietary communication protocols.

In one or more embodiments, the feedback component 602 can receive or facilitate receiving the various types of input data 402 described herein. In this regard, the feedback component 602 can receive sensory feedback data 404 from the sensor device 106 regarding usage of the absorbent article 102 and/or activity of the wearer. In some implementations, the received sensory feedback data can comprise processed feedback data providing various input parameters/parameter values related to usage and/or activity for input into the one or more fit evaluation algorithms/models and/or forecasting models.

In other embodiments, the received sensory feedback data can include raw sensor data (e.g., sensor measurements). With these embodiments, the feedback component 602 can be configured to process the raw sensor data using the sensor data to determine the corresponding usage and activity information. For example, in some embodiments, the sensor device 106 can be configured to send some or all of the sensor data (e.g., raw sensor data and/or digital information corresponding to captured/detected sensor measurement values) to the external user device 108 for processing and/or presentation by the external user device 108. The sensor data can include any measurements, properties, characteristics mentioned above with respect to sensory data feedback. With these embodiments, the feedback component 602 can be configured to process and analyze some or all sensor data to determine and/or infer sensory feedback data based on captured sensor measurements. For example, the feedback component 602 can process the sensor data to generate input parameters for input to the one or more fit evaluation algorithms/models and/or the one or more forecasting algorithms/models as discussed above.

The feedback component 602 can also receive and/or facilitate receiving the additional input information described herein, including but not limited to, wearer data 406, user feedback data 408 and auxiliary monitoring device feedback data 410. In some embodiments, the feedback component 602 can process this additional input data as appropriate to generate the corresponding information for usage by the fit evaluation component 604. For example, in implementations in which the additional feedback comprises image data of the wearer captured by the auxiliary monitoring device 302 and/or the one or more cameras 628, the feedback component 602 can process the image data using one or more image analysis techniques to determine information regarding fit of the absorbent article (e.g., relative positions and proportions of the absorbent article parts to the body of the wearer, level of sagging, location and severity of wear marks, etc.), activity of the wearer, behaviors of the wearer, mood of the wearer, and the like.

In some embodiments, the feedback component 602 can also determine input parameters/parameter values for input into the one or more fit evaluation and/or upgrade forecasting models based on a combination of received sensory feedback data 404, wearer data 406, user feedback data 408 and/or the auxiliary monitoring device feedback data 410. For example, the feedback component 602 can determine a status of the absorbent article based at least in part upon the sensory feedback data 404 received from the sensor device 106 and contextual information. Contextual information, for example, can be input by consumers, retrieved via other sensors or information sources (e.g., thermostats). For example, the data can include a property change in an indicator and wear time. Wear time, for example, may be described as the time determined between attachments of two fresh diapers. In various implementations, the feedback component 602 can determine and/or infer exudate fullness using one or more of the following functions: (Urine Fullness=f(Property Change Detection, wear time, wearer data and other data)); and Property Change Detection=f(color sensor data).

The fit evaluation component 604 can provide for evaluating the appropriateness of the fit of the absorbent article 102 for the wearer based on the various input information (e.g., input data 402) received by and/or generated by the feedback component 602 and/or the feedback component 602. In the embodiment shown, the fit evaluation component 604 can include sizing component 606, style/type evaluation component 608, upgrade forecasting component 610, and machine learning component 612.

In one or more embodiments, the sizing component 606 can be configured to evaluate the appropriateness of the current size of the absorbent article for the wearer. For example, with reference again to FIG. 4 and FIG. 6, at 414 the sizing component 606 can evaluate the appropriateness of the size based on the input data 402 using one or more fit evaluation algorithms/models. In this regard, the sizing component 606 can apply one or more fit evaluation algorithms/models to the input data 402, wherein the one or more fit evaluation algorithms/models are configured to generate an output regarding the appropriateness of the current size of the absorbent article for the wearer. The output may include binary indications and/or degree of appropriateness as discussed above. In some embodiments, the one or more fit evaluation algorithms/models can include one or more algorithms/models configured to determine, and/or generate an output identifying, an appropriate or optimal size absorbent article for wear by the person based on the input data 402 (e.g., the analysis performed at 420). With these embodiments, the sizing component 606 can determine whether the current size article worn is appropriate based on whether the current size matches the optimal size.

The style/type evaluation component 608 can similarly evaluate the appropriateness of the current style/type of the absorbent article for the wearer. For example, with reference again to FIG. 4 and FIG. 6, at 414, the style/type evaluation component 608 can evaluate the appropriateness of the style/type of the absorbent article based on the input data 402 using one or more fit evaluation algorithms/models. In this regard, the style/type evaluation component 608 can apply one or more fit evaluation algorithms/models to the input data 402, wherein the one or more fit evaluation algorithms/models are configured to generate an output regarding the appropriateness of the current style/type of the absorbent article for the wearer. The output may include binary indications and/or degree of appropriateness as discussed above. In some embodiments, the one or more fit evaluation algorithms/models can include one or more algorithms/models configured to determine, and/or generate an output identifying, an appropriate or optimal style/type of absorbent article for wear by the person based on the input data 402 (e.g., the analysis performed at 422). For example, the different styles/types of the absorbent article can vary by cut, shape, fit, closures, leg openings/fittings, absorbency level, material (e.g., hypoallergenic materials), type of indicators included, position of the indicators, perfumes and deodorants included, and the like. With these embodiments, the style/type evaluation component 608 can determine whether the current style/type of article worn by the person is appropriate based on whether the current style/type matches the optimal style/type.

In this regard, the one or more size fit evaluation algorithms/models can include heuristic based algorithms/models, statistical-based algorithms/models, probabilistic-based algorithms models, and/or machine learning algorithms/models. For example, the sizing component 606 and/or the style/type evaluation component 608 can evaluate the input data 402 to determine information regarding appropriateness of the absorbent article size and/or style/fit for the wearer using fuzzy logic techniques, SVMs, Hidden Markov Models (HMMs), greedy search algorithms, rule-based systems, Bayesian models (e.g., Bayesian networks), neural networks, other non-linear training techniques, data fusion, utility-based analytical techniques, Bayesian models, classification models, complex heuristic models, and the like.

The upgrade forecasting component 610 can be configured to apply one or more upgrade forecasting algorithms/models to the input data 402 to determine and/or infer when a person is expected to need a larger size absorbent article based on the input data 402 and/or when the person is expected to need a different style/type of absorbent article based on the input data 402. For example, in various embodiments, the upgrade forecasting component 610 can apply one or more size upgrade forecasting models to the input data 402 to forecast when (e.g., a number of days, weeks, months, etc., from a current point in time) a person will outgrow their current absorbent article size. In some implementations, the one or more size upgrade forecasting models can also forecast the specific size that the person will need to upgrade to (e.g., which could be the next size up or a different size). In some embodiments, the upgrade forecasting component 610 can also apply one or more style/type upgrade forecasting models to the input data 402 to forecast when (e.g., a number of days, weeks, months, etc., from a current point in time) a person will outgrow their current absorbent article style/type and/or otherwise need a different absorbent article style/type. In some implementations, the one or more style/type upgrade forecasting models can also forecast the specific style/type that the person will need to upgrade to. In this regard, the one or more size upgrade forecasting models and/or the one or more style/type upgrade forecasting models can include heuristic based algorithms/models, statistical-based algorithms/models, probabilistic-based algorithms models, and/or machine learning algorithms/models. Some suitable machine learning models that can be used for the upgrade forecasting models can include but are not limited to: a support vector machine model, a linear regression model, a logistic regression model, a naïve Bayes model, a linear discriminant analysis model, a decision tree model, a k-nearest neighbor model, a neural network model, and the like.

The machine learning component 612 can perform various machine learning techniques to facilitate determining or inferring whether a current size and/or style/type of absorbent article worn by a person is appropriated for the person, a particular size and/or style/type of absorbent article that from amongst size and/or style/type options that is most appropriate for the person, and/or forecasting when a person is expected to need a different size and/or style/type of absorbent article (and to what size and/or type). In general, machine learning is concerned with the design and the development of techniques that take as input empirical data (such as network statistics and performance indicators) and recognize complex patterns in the data. A common pattern among machine learning techniques is the use of an underlying model M, whose parameters are optimized for minimizing the cost function associated to M, given the input data. For instance, in the context of classification (e.g., classification of an absorbent article as appropriate or inappropriate, classification of an absorbent article as too big, too large, or just right, etc.), the model M may be a straight line that separates the data into two classes (e.g., labels) such that M=a*x+b*y+c and the cost function would be the number of misclassified points. The learning process then operates by adjusting the parameters a, b, c such that the number of misclassified points is minimal. After this optimization phase (or learning phase), the model M can be used very easily to classify new data points. Often, M is a statistical model, and the cost function is inversely proportional to the likelihood of M, given the input data.

Computational entities that rely on one or more machine learning techniques to perform a task for which they have not been explicitly programmed to perform are typically referred to as learning machines. In particular, learning machines are capable of adjusting their behavior to their environment. For example, a learning machine may dynamically make future predictions based on current or prior network measurements, may make control decisions based on the effects of prior control commands, etc.

In this regard, in some embodiments, the machine learning component 612 can regularly and/or continuously update and/or optimize the one or more fit evaluation models and/or upgrade forecasting models based on the received input data for a specific user over time. In some embodiments in which the machine learning component 612 is executed by the server device 502, the machine learning component 612 can further perform model updating and optimization based on feedback data collected for many different users over time (e.g., as discussed with reference to FIG. 5). For example, as described above, one or more given fit evaluation models and/or upgrade forecasting models (e.g., a supervised, un-supervised, or semi-supervised model) can be used to generate information regarding appropriateness of size and/or style/type and/or information regarding when a person is expected to outgrow a current size and/or style/type. Example machine learning techniques that may be used to construct and analyze these models can include, but are not limited to, nearest neighbor (NN) techniques (e.g., k-NN models, replicator NN models, etc.), statistical techniques (e.g., Bayesian networks, etc.), clustering techniques (e.g., k-means, etc.), neural networks (e.g., reservoir networks, artificial neural networks, etc.), support vector machines (SVMs), or the like.

In one example, at least one fit evaluation model and/or upgrade forecasting model used by the fit evaluation component 604 can be a classification machine learning model that maps the input data 40 to one or more categories. In another example, at least one fit evaluation model and/or upgrade forecasting model used by the fit evaluation component 604 can be a regression machine learning model that determines relationships amongst various parameters included in the input data 402. In yet another example, at least one fit evaluation model and/or upgrade forecasting model used by the fit evaluation component 604 can be a clustering machine learning model that groups related data included in the input data 402 (e.g., for a single wearer and/or for many different wearers) into a corresponding group.

In an embodiment, the machine learning component 612 can employ one or more artificial intelligence techniques to execute and/or facilitate execution of (e.g., by the sizing component 606, the style/type evaluation component 608 and/or the upgrade forecasting component 610) of the one or more fit evaluation models and/or upgrade forecasting models based on the input data 402. For example, the machine learning component 612 can extract information that is indicative of correlations, inferences and/or expressions from the input data 402 based on principles of artificial intelligence. The machine learning component 612 can generate the machine learning output based on the execution of the one or more fit evaluation models and/or upgrade forecasting models based on the input data 402. The machine learning output can include, for example, learning, correlations, inferences and/or expressions associated with the input data 402. In an aspect, the machine learning component 612 can perform learning with respect to the input data 402 explicitly or implicitly. The machine learning component 612 can also employ an automatic classification system and/or process to facilitate analysis of the input data 402. For example, the machine learning component 612 can employ a probabilistic and/or statistical-based analysis (e.g., factoring into the analysis utilities and costs) to learn and/or generate inferences with respect to the input data 402. The machine learning component 612 can employ, for example, a support vector machine (SVM) classifier to learn and/or generate inferences for the input data 402. Additionally, or alternatively, the machine learning component 612 can employ other classification techniques associated with Bayesian networks, decision trees and/or probabilistic classification models. Classifiers employed by the machine learning component 612 can be explicitly trained (e.g., via a generic training data) as well as implicitly trained (e.g., via receiving extrinsic information). For example, with respect to SVM's, SVM's can be configured via a learning or training phase within a classifier constructor and feature selection module. A classifier can be a function that maps an input attribute vector, x=(x1, x2, x3, x4, xn), to a confidence that the input belongs to a class—that is, f(x)=confidence(class).

In an aspect, the machine learning component 612 can include an inference component (not shown) that can further enhance automated aspects of the machine learning component 612 utilizing in part inference-based schemes to facilitate learning and/or generating inferences for the input data 402. The machine learning component 612 can employ any suitable machine-learning based techniques, statistical-based techniques and/or probabilistic-based techniques, such as the various examples discussed above. In another aspect, the machine learning component 612 can perform a set of machine learning computations associated with analysis of the input data 402. For example, the machine learning component 612 can perform a set of clustering machine learning computations, a set of logistic regression machine learning computations, a set of decision tree machine learning computations, a set of random forest machine learning computations, a set of regression tree machine learning computations, a set of least square machine learning computations, a set of instance-based machine learning computations, a set of regression machine learning computations, a set of support vector regression machine learning computations, a set of k-means machine learning computations, a set of spectral clustering machine learning computations, Gaussian mixture model machine learning computations, a set of regularization machine learning computations, a set of rule learning machine learning computations, a set of Bayesian machine learning computations, a set of deep Boltzmann machine computations, a set of deep belief network computations, a set of convolution neural network computations, a set of stacked auto-encoder computations and/or a set of different machine learning computations.

The recommendation component 614 can further provide recommendations to users based on the results of the fit evaluation analysis performed by the fit evaluation component 604. For example, in some implementations, the recommendation component 614 can recommend a wearer change their current size absorbent article to a different size based on a determination that the current size is inappropriate (e.g., too big or too small). The recommendation can also include information identifying the best (most appropriate) size absorbent article determined for wear by the person. Similarly, the recommendation component 614 can recommend a wearer change their current style/type absorbent article to a different style/type based on a determination that the style/type is inappropriate or otherwise not the most appropriate option for the wearer. The recommendation can also include information identifying the best (most appropriate) style/type of absorbent article. The recommendation component 614 can also provide users with recommendations and/or notifications regarding forecasted size and/or style/type upgrades. For example, the recommendation component 614 can generate a recommendation or notification that informs a user when the wearer 101 is expected to need a larger size article and/or a different style/type of article. In some examples, the recommendation component can inform a user of different products, sizes, styles available from one or more manufacturers and/or retailers.

In some embodiments, the ordering component 616 can facilitate ordering additional absorbent articles for use with the sensor device 106 based on the recommendations determined by the recommendation component 614 and/or tracked usage. For example, in one or more embodiments, the ordering component 616 can automatically order additional absorbent articles in a recommended size and/or style/type based on a determination that the current size and/or style/type is inappropriate for the wearer. In some embodiments, the ordering component 616 can also order additional absorbent articles in a different size and/or style/type ahead of time based on forecasted time when the recommendation component recommends the wearer upgrade to the different size and/or style/type. For example, in some implementations, the user can participate in an automatic refill or ordering program wherein the user receives additional disposable diapers (or another type of absorbent article) once a week, once a month, etc. In accordance with this example, the ordering component 616 can employ the forecasted upgrade information to determine what size and/or style/type of articles to order each week, month, etc. and optionally what quantity of articles. For instance, assuming the user receives N amount of diapers once a month and next month, the user's baby is expected to need a larger size diaper, the ordering component 616 can automatically order the larger size diaper for delivery next month.

The ordering component 616 can further facilitate automatically ordering the additional product based on the tracked usage. In some embodiments, the reward component 618 can be configured to issues a reward to an entity account associated with the sensor device 106 and/or external user device 108 based ordering of the additional absorbent articles using with an authorized merchant. In other embodiments, the reward component 618 can provide the external user device 108 and/or a user account with reward based on various other types of user activity of the connected care system/application. For example, such other user activity can include but is not limited to: providing the server device 502 with collected input data 402, using interactive and/or social media features associated with the application, providing manual input regarding behavior of the wearer, wearer habits (e.g., feeding habits, moods, etc.), appearance of the wearer, and other relevant data about the wearer. In some embodiments, the reward component 618 can also provide the user/user account with rewards for allowing the system to access tracked data about the wearer provided by the sensor device 106, and other data received and/or generated via the usage of the connected care application 600. Rewards may include a coupon, a discount, a credit, reward points, free shipping, additional features of the connected care application and/or sensor device, ancillary services, products and combinations thereof.

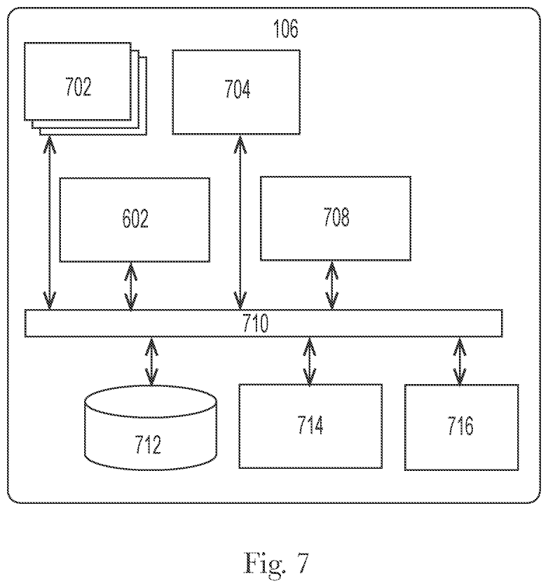

With reference now to FIG. 7, illustrated is a block diagram of an example sensor device (e.g., sensor device 106). Various aspects of devices, systems, apparatuses or processes explained in this disclosure can constitute machine-executable component(s) embodied within machine(s), e.g., embodied in one or more computer readable mediums (or media) associated with one or more machines. Such components, when executed by the one or more machines, (e.g., computers, computing devices, virtual machines, etc.) can cause the machines to perform the operations described.

For example, in the embodiment shown, the sensor device 106 can include one or more sensors 702, communication component 704, feedback component 602, and notification component 708, which can respectively be or include computer/machine-executable components and instructions. The sensor device 106 can also include at least one memory 712 configured to store the computer/machine-executable components and instructions. The sensor device 106 can also include at least one processor 714 (e.g., a microprocessor) to facilitate operation of the computer executable components and instructions by the sensor device 106. The sensor device 106 can further include a power unit 716 that provides for powering the various electrical components of the sensor device 106. In various embodiments, the sensor device 106 can incorporate a non-rechargeable battery as the power unit 716. However, other suitable type of power sources/units are envisioned. The sensor device 106 can further include a device bus 710 that couples the various components of the sensor device 106, including, but not limited to: the one or more sensors 702, the communication component 704, the feedback component 602, the notification component 708, the memory 712, the processor 714 and the power unit 716.

In various embodiments, the one or more sensors 702 formed on or within a housing of the sensor device (e.g., housing 202) and provide for sensing, detecting or otherwise capturing sensor data regarding usage of an absorbent article to which the sensor device 106 is designed to be attached, and/or activity of the wearer. For example, as discussed with reference to FIGS. 1, 2A and 2B, in various embodiments, the one or more sensors 702 can include one or more optical sensors configured to detect information regarding article usage based on optical responses/reactions reflected in the one or more indicators 112 provided on or within the absorbent article 102. Some examples of optical sensors across a range of wavelengths are: electron tube detectors, photosensors, photomultiplier tubes, phototubes, photodetectors, opto-semiconductor detectors, photodiodes, photomultipliers, image sensors, infrared detectors, thermal sensors, illuminance sensors, visible light sensors and color sensors. In some implementations, the sensor device 106 can also include a light source (e.g., light source 208), such as a light emitting diode (LED), organic light emitting diode (OLED), an incandescent light bulb, thermionic light emission, luminescence (e.g., among others, fluorescence, chemilluminescence, electroluminescence (e.g., LED), for emitting light onto an area, the wavelength or spectrum of which is to be assessed by the optical sensor. The optical sensor in some color detecting embodiments can be optimized for assessing a color of a color-changing indicator. The optical sensor can be sensitive to visible and non-visible light. In various embodiments, ultraviolet (UV), visible infrared and near infrared wavelengths may be used.

The one or more sensors 702 can include one or more activity sensors configured to sense, detect, or otherwise capture information regarding activity of a wearer of the absorbent article to which the sensor device 106 is attached. For example, the one or more activity sensors can include one or more motion/movement sensors (e.g., an accelerometry, a gyroscope, and the like) that can capture motion data which can be correlated to defined bodily movements and/or motions, activity levels, activity patterns, sleep patterns, and the like. In some embodiments, the one more sensors 702 can also include biofeedback sensors configured to detect physiological parameters associated with the wearer of the sensor device 106, including heart rate, temperature, and other vital signs, biomarkers present in bodily exudates, and the like. The one or more sensors 702 can also include also one or more sensors configured to sense, detect or otherwise capture sensor data reflective on a behavior of the wearer of the sensor device 106/absorbent article 102, pressure and irritation associated with fit and wear of the absorbent article, and the like. In this regard, in various embodiments, the one or more sensors 702 employed by the sensor device 106 can include but are not limited to one or more: image sensors, optical sensors, chemical sensors, biosensors, a biochemical sensors, temperature sensors, force/pressure sensors, motion sensors (e.g., an accelerometer, a gyroscope, etc.), humidity sensors, acoustic sensors, an RFID reader/sensor, and the like.

The communication component 704 can provide for communicatively coupling the sensor device 106 with one or more external devices, such as external user device 108, auxiliary monitoring device 302, server device 502 and/or various other devices remote (e.g., physically remote) from sensor device 106. The communication component 704 can include same or similar features and functionalities as communication component 626. Repetitive description of like elements employed in respective embodiments is omitted for sake of brevity.

In the embodiment shown, the sensor device 106 can also include feedback component 602 to facilitate processing raw sensor data captured via the one or more sensors 702. For example, in some embodiments, the sensor device 106 can be configured to send some or all sensor data (e.g., raw sensor data and/or digital information corresponding to captured/detected sensor measurement values) to the external user device 108 for processing and/or presentation by the external user device 108. The sensor data can include any measurements, properties, characteristics mentioned above with respect to sensory data and feedback. The external user device 108 can be configured to process and analyze some or all sensor data to determine and/or infer sensory feedback data based on captured sensor measurements.

Additionally, or alternatively, the feedback component 602 can be configured to provide for partial and/or full onboard processing of the sensor data to generate the sensory feedback data (e.g., the input parameters) based on the captured sensor data. For example, in various embodiments, the feedback component 602 can be configured to process/analyze the sensor data captured via the one or more sensors using predefined processing logic (e.g., algorithms, heuristics, machine learning models, defined correlations, tracked data correlations, etc.) to determine and/or infer sensory feedback data regarding usage and/or activity information for the wearer.

For example, in one or more implementations, an optical sensor of the sensor device 106 can detect and/or determine one or more RGB light levels or HSB levels of a color strip indicator provided within the absorbent article. The feedback component 602 can further determine and/or infer sensory feedback data regarding usage (e.g., presence, absence and/or amount of one or more bodily exudates, and/or a local saturation), based at least in part on the RGB light level or HSB level information as captured/generated by the one or more sensors 702. In another example, the one or more sensors 702 can include sensors that capture activity data (e.g., motion sensors), and the feedback component 602 can be configured to process the activity data to determine sensory feedback data regarding activity levels and/or patterns of the wearer, and the like based on the captured activity data. In some implementations, the feedback component 602 can further provide for determining and/or inferring additional information regarding the behavior of the wearer, and the physiological and/or health status of person based on the captured sensor data (e.g., detected properties of the bodily exudates, timing and frequency of the bodily exudates, activity level/patterns of the wearer over time, and/or various other captured and/or tracked parameters).

In some implementations, the communication component 704 can be configured to send the determined/inferred sensory feedback data to the external user device 108 for rendering and/or further processing by the external user device 108 (e.g., to perform the fit evaluation and/or size change forecasting analysis) and/or forwarding to the server device 502 (or another device). The external user device 108 can further be configured to present or otherwise provide the determined information to the user as a real-time notification, as an assessment report, or the like.

In various implementations, the notification component 708 can be configured to generate and send notifications to the external user device 108 based on detection of defined sensor measurement values and/or based on a determination, by the feedback component 602, that a defined event or condition has occurred as determined based on the sensor data. By way of nonlimiting example, the notification component 708 can be configured to generate and send the external user device 108 a notification that the absorbent article is wet based on a determination that the absorbent article is wet or has reached a defined saturation level. In another nonlimiting example, the notification component 708 can generate and send the external user device 108 a notification that wearer has woken based on a determination that the wearer's activity level/pattern indicates the wearer is no longer asleep.

In some embodiments, the sensor device 106 can further include one or more of the other components associated with the connected care application 600 discussed with reference to FIG. 6. For example, the sensor device can include one or more of, the fit evaluation component 604, the recommendation component 614, the ordering component 616, and/or the reward component. Additionally, or alternatively, one or more of these components can be included with and/or executed by the server device 502.

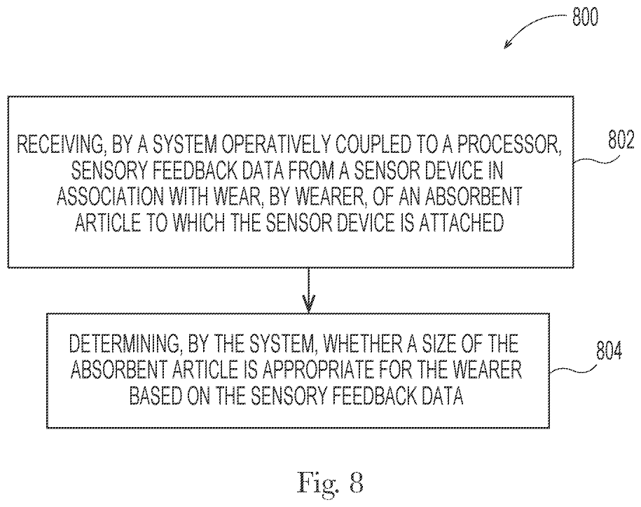

FIG. 8 presents a high-level flow diagram of another example process 800 for determining whether to change the size of a person's absorbent articles accordance with one or more embodiments of the disclosed subject matter. Repetitive description of like elements employed in respective embodiments is omitted for sake of brevity.

At 802, a system operatively coupled to a processor (e.g., system 100, system 300, system 500 and the like) can receive sensory feedback data (e.g., sensory feedback data 404) from a sensor device (e.g., sensor device 106) in association with wear of an absorbent article to which the sensor device is attached (e.g., using feedback component 602). At 804, the system can determine whether a size of the absorbent article is appropriate for the person based on the sensory feedback data (e.g., using sizing component 606).

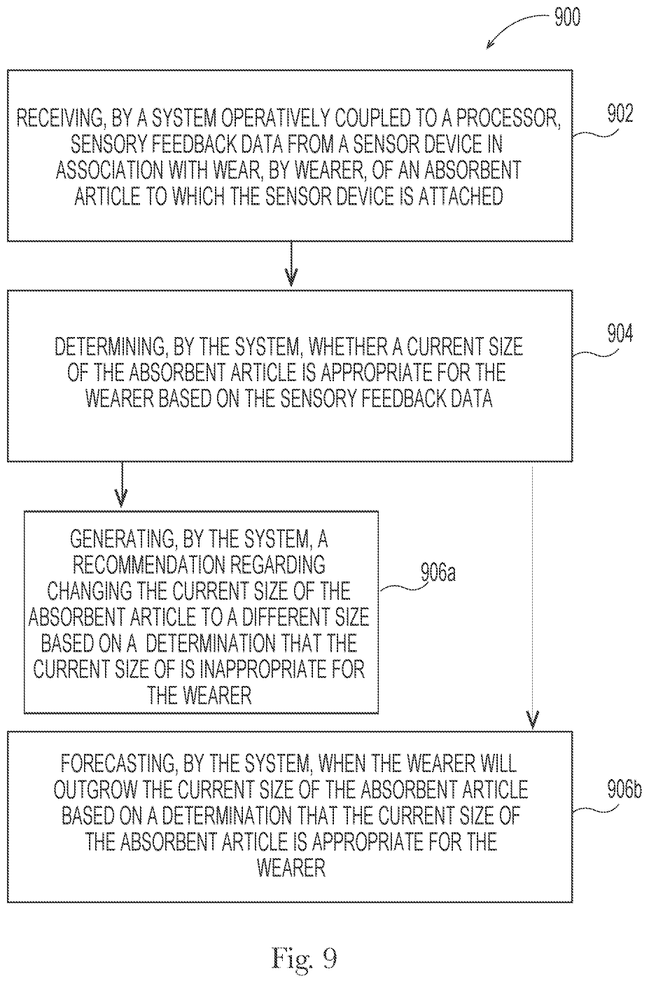

FIG. 9 presents a high-level flow diagram of another example process 900 for determining whether to change the size of a person's absorbent article accordance with one or more embodiments of the disclosed subject matter. Repetitive description of like elements employed in respective embodiments is omitted for sake of brevity.

At 902, a system operatively coupled to a processor (e.g., system 100, system 300, system 500 and the like) can receive sensory feedback data (e.g., sensory feedback data 404) from a sensor device (e.g., sensor device 106) in association with wear of an absorbent article to which the sensor device is attached (e.g., using feedback component 602). At 904, the system can determine whether a current size of the absorbent article is appropriate for the person based on the sensory feedback data (e.g., using sizing component 606). At 906a, the system can generate a recommendation regarding changing the current size of the absorbent article to a different size based on a first determination that the current size is inappropriate for the person (e.g., using recommendation component 614). At 906b, the system can forecast when the person will outgrow the current size of the absorbent article based on a second determination that the current size is appropriate for the person (e.g., using upgrade forecasting component 610).

The flowcharts and block diagrams in the Figures illustrate the architecture, functionality, and operation of possible implementations of systems, methods, and computer program products according to various embodiments of the present invention. The functions noted in the blocks can occur in the order shown in the Figure(s) or out of the order noted in the Figure(s), depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

While the subject matter has been described above in the general context of computers, those skilled in the art will recognize that this disclosure also can or can be implemented in combination with other program modules. Generally, program modules include routines, programs, components, data structures, etc. that perform particular tasks and/or implement particular abstract data types. Moreover, those skilled in the art will appreciate that the inventive computer-implemented methods can be practiced with other computer system configurations, including single-processor or multiprocessor computer systems, mini-computing devices, mainframe computers, as well as computers, hand-held computing devices (e.g., PDA, phone), microprocessor-based or programmable consumer or industrial electronics, and the like. The illustrated aspects can also be practiced in distributed computing environments in which tasks are performed by remote processing devices that are linked through a communications network. However, some, if not all aspects of this disclosure can be practiced on stand-alone computers. In a distributed computing environment, program modules can be located in both local and remote memory storage devices.

In addition, the term “or” is intended to mean an inclusive “or” rather than an exclusive “or.” That is, unless specified otherwise, or clear from context, “X employs A or B” is intended to mean any of the natural inclusive permutations. That is, if X employs A; X employs B; or X employs both A and B, then “X employs A or B” is satisfied under any of the foregoing instances. Moreover, articles “a” and “an” as used in the subject specification and annexed drawings should generally be construed to mean “one or more” unless specified otherwise or clear from context to be directed to a singular form.

The descriptions of the various embodiments have been presented for purposes of illustration, but are not intended to be exhaustive or limited to the embodiments disclosed. Many modifications and variations will be apparent to those of ordinary skill in the art without departing from the scope and spirit of the described embodiments.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm.”

Every document cited herein, including any cross referenced or related patent or application, is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A system comprising:
a processor that executes computer executable components, the computer executable components comprising:
a feedback component that receives sensory feedback data from a sensor device in association with wear, by a wearer, of an absorbent article to which the sensor device is attached; and
a sizing component that determines whether a size of the absorbent article is appropriate for the wearer based on the sensory feedback data.

2. The system of claim 1, wherein the computer executable components further comprise: a recommendation component that generates a recommendation regarding changing the size of the absorbent article to a different size based on a determination that the size of the absorbent article is inappropriate for the wearer.

3. The system of claim 2, wherein the sizing component further determines an appropriate size of the absorbent article for the wearer based on the sensory feedback data, and wherein the recommendation comprises information identifying the appropriate size.

4. The system of claim 1, wherein the sensory feedback data comprises activity information of the wearer in association with wear of the absorbent article.

5. The system of claim 4, wherein the activity information comprises sleep pattern information.

6. The system of claim 1, wherein the sensory feedback data comprises usage information.

7. The system of claim 6, wherein the usage information comprises at least one of the group consisting of: amount of the bodily exudates associated with an exudation event, saturation levels, time to saturation, amounts of bodily exudates contained with the absorbent article over a period of time, frequency of exudates events, and frequency of changes of absorbent articles.

8. The system of claim 1, wherein sizing component further determines whether the size of the absorbent article is appropriate for the wearer based on one or more attributes of the wearer selected from a group consisting of: an age of the wearer, a weight of the wearer, and a height of the wearer.

9. The system of claim 1, wherein the feedback component further receives user feedback data, and wherein the sizing component further determines whether the size of the absorbent article is appropriate for the wearer based on the user feedback data.

10. The system of claim 1, wherein the user feedback data comprises user input regarding wear marks.

11. The system of claim 1, wherein sizing component employs one or more machine learning techniques to determine whether the size of the absorbent article is appropriate for the wearer based on the sensory feedback data.

12. The system of claim 1, wherein the computer executable components further comprise:
an ordering component that generates an order for new absorbent articles for an entity account associated with the sensor device.

13. A method comprising:
receiving, by a system operatively coupled to a processor, sensory feedback data from a sensor device in association with wear, by a wearer, of an absorbent article to which the sensor device is attached; and
determining, by the system, whether a size of the absorbent article is appropriate for the wearer based on the sensory feedback data.

14. The method of claim 13, further comprising:
generating, by the system, a recommendation regarding changing the size of the absorbent article to a different size based on a determination that the size of the absorbent article is inappropriate for the wearer.

15. The method of claim 13, wherein the sensory feedback data comprises activity information regarding the wearer in association with wear of the absorbent article.

16. The method of claim 15, wherein the activity information comprises sleep pattern information.

17. The method of claim 13, wherein the sensory feedback data comprises usage information.

18. The method of claim 17, wherein the usage information comprises at least one of the group consisting of: amount of the bodily exudates associated with an exudation event, saturation levels, time to saturation, amounts of bodily exudates contained with the absorbent article over a period of time, frequency of exudates events, and frequency of changes of absorbent articles.

19. A machine-readable storage medium comprising executable instructions that, when executed by a processor of a device, facilitate performance of operations, comprising:
receiving sensory feedback data from a sensor device in association with wear, by a wearer, of an absorbent article to which the sensor device is attached;
determining whether a size of the absorbent article is appropriate for the wearer based on the sensory feedback data; and
generating a recommendation regarding changing the size of the absorbent article to a different size based on a determination that the size of the absorbent article is inappropriate for the wearer.

20. The machine-readable storage medium of claim 19, wherein the sensory feedback data comprises one or more of: activity information regarding activity patterns of the wearer in association with the wear of the absorbent article, and usage information regarding at least one of: an amount of bodily exudates received within the absorbent article, frequency of generation of the bodily exudates by the wearer, frequency of changes of the absorbent article, and time to saturation of the absorbent article, and wherein the operations further comprise:
employing one or more machine learning techniques to determine whether the size of the absorbent article is appropriate for the wearer based on the sensory feedback data.
